(19) United States
(12) Patent Application Publication
(10) Pub. No.: US 2024/0108535 A1
(43) Pub. Date: Apr. 4, 2024

(54) A THERAPEUTIC MASSAGE DEVICE

(71) Applicant: Bradley CARDEN, Manly Vale (AU)

(72) Inventor: Bradley CARDEN, Manly Vale (AU)

(21) Appl. No.: 18/264,648

(22) PCT Filed: Mar. 3, 2022
(86) PCT No.: PCT/AU22/50175

§ 371 Date: Aug. 8, 2023

(30) Foreign Application Priority Data
Mar. 4, 2021 (AU) 2021900608

(51) Int. Cl. A61H 15/02

(52) U.S. Cl. CPC A61H 15/02; A61H 2015/0014

(57) ABSTRACT

A massage device has a generally cylindrical body comprising an internal battery and controller for a plurality of light source elements. The body defines an exterior surface comprising the light source elements arranged in a matrix to emit light from a surface area of the surface.

### FIELD OF THE INVENTION

This invention relates generally to a therapeutic massage device. More particularly, this invention relates to a therapeutic massage device which applies simultaneous therapeutic photobiomodulation (PBM) in the form of red or near-infrared (NIR) light to stimulate, heal, regenerate damaged tissue and accelerate recovery.

### SUMMARY OF THE DISCLOSURE

There is provided herein a therapeutic massage device which applies Photobiomodulation (PBM) in the form of red or near-infrared (NIR) light in addition to massage pressure to stimulate, heal, and regenerate damaged tissue.

The present device may be used both for preconditioning (therapeutic light delivered to muscles before exercise) and after exercise to improve skeletal muscle recovery, increase sports performance in athletes and the like.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows an exemplary control system of massage device in accordance with an embodiment;

FIG. 2 shows a perspective view of the massage device in accordance with an embodiment;

FIG. 3 shows a magnified cross-sectional view of the device in accordance with an embodiment; and

FIG. 4 illustrates the assembly of a cylindrical core and flexible sleeve of the device in accordance with an embodiment.

### DESCRIPTION OF EMBODIMENTS

A therapeutic massage device 100 may comprise a generally cylindrical body 101 comprises an internal rechargeable battery 102 and may comprise a controller 103 for a plurality of light source elements 104. The light source elements 104 may comprise LEDs.

The body 101 defines an exterior surface 105 and the light source elements 104 may be arranged in a matrix 106 to emit light from the exterior surface 105. The light source elements may emit red and near infrared light in the range of 600 nm-1000 nm.

The controller 103 may control switching to control the light source elements 104 to control the operation of the light source elements 104 on or off. Furthermore, the controller 103 may control the intensity or frequency of the light source elements 104. In embodiments, the light source elements 104 may be controlled by the controller 103 to pulsate.

The spacing and intensity of the light source elements may provide a power density of between 0.005 W/cm² and 5 W/cm² across the exterior surface 105.

In embodiments, the body 101 comprises a diameter greater than 10 cm and a length greater than 20 cm, such as approximately 30 cm.

The light source elements 104 preferably extend across at least 80% of the length of the body 101.

Whereas the light source element 104 are shown as being equidistantly spaced across the surface 105 in FIG. 4, in embodiments, the density of the light source elements 104 may increase towards the centre of the length of the body 101 to target muscle tissue located centrally on the surface 105.

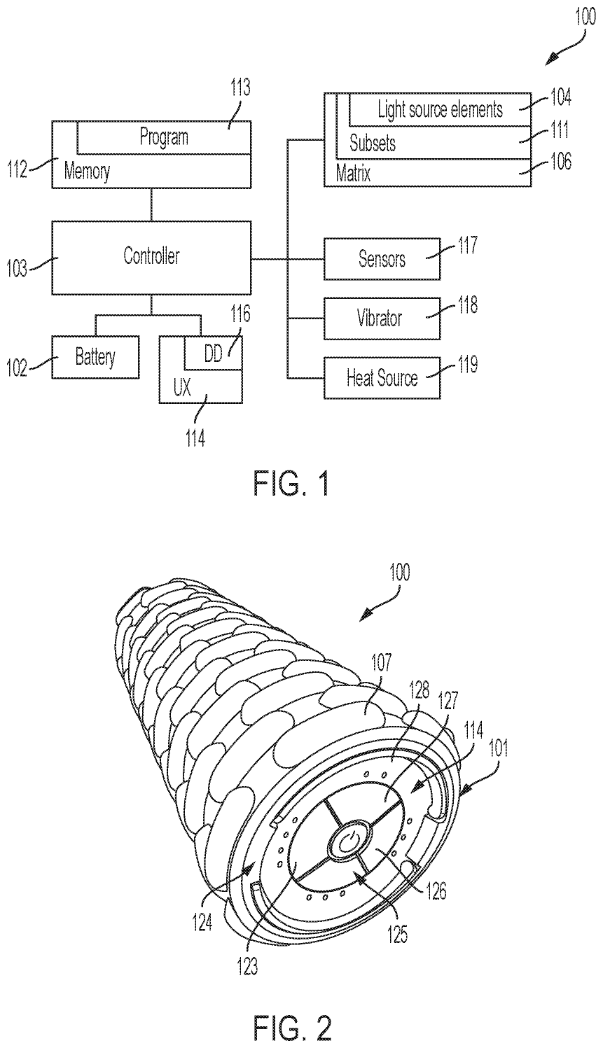

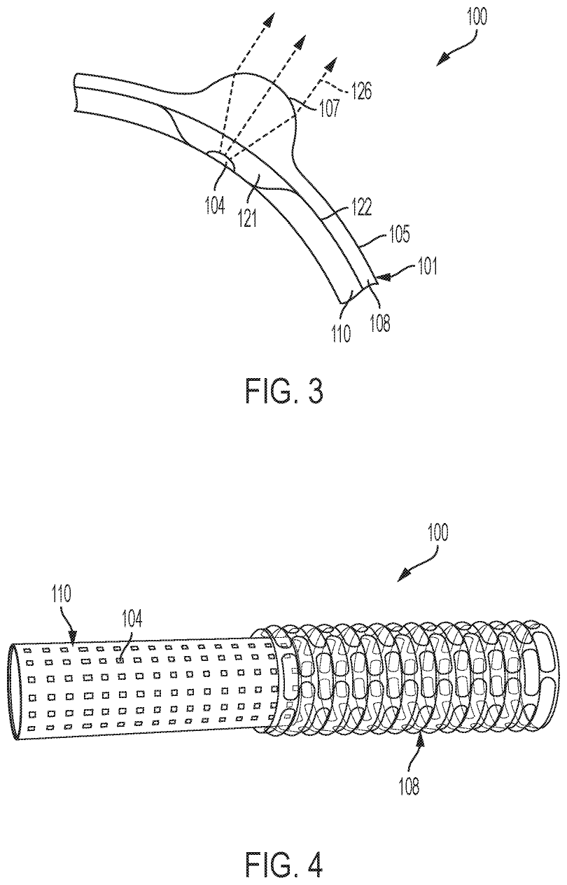

With reference to FIGS. 2 and 3, the surface 105 may comprise a plurality of massage protrusions 107. The light source elements 104 may collocate with respective protrusions 107 to thereby provide coincident photobiomodulation therapy and massage pressure to the same location of the skin.

With reference to FIGS. 3 and 4, the device 100 may comprise a rigid core 110 and a transparent sleeve 108. The transparent sleeve 108 is preferably flexible and may be made of transparent silicon.

FIG. 4 shows how the transparent sleeve 108 may slide over the rigid core 110 to assemble the device 100.

As shown in FIG. 3, each light source element 104 may be recessed in a recess 121 beneath an exterior surface 122 of the cylindrical core 110. The recess 101 may be narrower than the adjacent massage protrusion 107 to prevent collapsing of the intrusion 107 under pressure.

In embodiments, the interior surface area of the recess 121 may comprise reflective material to reflect light from the light source element 104 outwardly. In further embodiments, the surface area 122 of the cylindrical core 110 away from the recesses 121 further comprises a reflective material so as to reflect back to light reflecting from the skin.

FIG. 3 illustrates the protrusions 107 shaped as a plano-convex lens to focus light 126 towards the skin. In the embodiment shown in FIG. 2, the protrusions 107 are elongate with respect to a tangential axis of the body 101 and are domed in cross section with respect to a longitudinal axis of the body 101.

Furthermore, the protrusions 107 may be arranged along a series of adjacent rings along the length of the body 101. Ends of protrusions 107 of adjacent rings may overlap.

Referencing FIG. 1, the light source elements 104 may comprise a plurality of subsets 111 of light source elements and the controller 103 may be able to control the switching to independently control the subsets 111 of light source elements 104.

In embodiments, the subsets 111 of light source elements 104 emit light in different wavelength ranges and the controller 103 is able to independently control the subsets 111 to emit light in different wavelength ranges. For example, the different wavelength ranges may centre at approximately 660 nm and 850 nm respectively which may be used for different therapeutic effect.

As shown in FIG. 1, the controller 103 may be in operable communication with a memory device 112 across a system bus. The memory device 112 is configured for storing digital data including computer program code instructions. In use, the controller 103 fetches these computer program code instructions and associated data from the memory device 102 for implementing the functionality described herein. The memory may store therapeutic program 113 which is used by the controller 103 to control the light source elements 104 accordingly.

In one embodiment, the therapeutic program 113 specifies a dosage parameter. As such, the controller 103 may control at least one of duration and intensity of the light source elements 104 according to the dosage parameter.

The program may further specify at least one of wavelength range and treatment frequency wherein the controller is controllable to control the light source elements according to these parameters.

The program 103 may be adjusted according to user settings such as muscle group and or a user-specified level.

For example, the program may specify that 10J of dosage is required for 30 minutes a day in the wavelength of 660 nm for calf muscles that are relatively sore after exercise (i.e., user-specified soreness level) whereas 15 J is required for 30 minutes a day in the wavelength of 660 nm followed by 15 minutes in the wavelength of 850 nm for calf muscles that are quite sore after exercise.

As shown in FIG. 1, the controller 103 may interface a user control interface 114. As shown in FIG. 2, the user interface 114 may locate at one end 115 of the body 101. The user interface 114 may comprise a digital display 116 for the display of digital information thereon. The user interface 114 may comprise one or more pushbuttons. Alternatively, the digital display 116 may comprise a haptic overlay to detect user interface gestures with reference to information displayed thereon.

FIG. 2 shows an exemplary pushbutton user interface 114 with LED status displays in accordance with an embodiment. The interface 114 may comprise an intensity control button 123 which may be pressed repeatedly to control light intensity. The current light intensity may be displayed by light intensity indicators 124 which, in embodiments shown, shows light intensity from 25 to 100%.

User interface 114 may further comprise a timer control button 125 which may be pressed repeatedly to control and operational time of the LEDs such as for 10, 15 or 20 minutes.

User interface 114 may further comprise a vibration speed control button to control the operation and speed of a vibratory device 118, referred to in further detail below.

Furthermore, the user interface 114 may further comprise a light frequency control button 127 which may be repeatedly pressed to control the intensity of light emitted by the light source elements 114. Light intensity indicators 128 may indicate the light frequency which may be generally categorised as red or infrared as shown (or 660 nm and 850 nm in embodiments).

In embodiments, subsets 111 of the light source elements 104 may be independently controllable by the controller 103 to emit light within radial angle ranges with respect to a longitudinal axis of the body 101. In other words, the device 100 may be controllable to emit light from one side of the surface 105 only. Emitting light within these discrete radial angle ranges may be used for various purposes, including only irradiating muscle in contact with the body 101, emitting light in a certain direction (such as wherein the device 100 is stood atop a table and used to direct light towards the user's body).

In embodiments, the device 100 comprises at least one sensor 117 in operable communication with the controller 103. The controller 103 may determine an operative radial angle range in accordance with sensor data received from the at least one sensor.

For example, the at least one sensor 117 may comprise a pressure sensor. In this regard, the device 100 may comprise a plurality of pressure sensors located across the surface area 105 underneath the exterior 108 of the body 101. These pressure sensors are able to detect areas of the surface area 105 where pressure is being applied.

As such, for example, when rolling the device 100 across a calf muscle, the pressure sensors may detect the pressure applied by the calf muscle so that the controller 103 may operate the light source elements 104 underneath the calf muscle only. The controller 103 may distinguish between pressure applied by the flat surface underneath the calf muscle by differentiating between the centrally applied pressure of the calf muscle as compared to the longitudinally uniform pressure applied by the flat surface.

In addition, the controller 103 may control the light source elements 104 along the length of the body 101 according to the detected pressure. In other words, where the calf muscle applies pressure in the middle of the surface 105 only, the controller 103 may control the light source elements 104 to apply light only at the middle of the surface 105.

In alternative embodiments, the sensor 107 may comprise an orientation sensor. The orientation sensor 107 may take the form of a solid-state integrated circuit orientation sensor. As such, the controller 103 may determine the orientation of the body 101 using the orientation sensor and control the subset 111 of light source element is 104 accordingly. For example, the controller 103 may be configured to control the light source elements 104 to emit light only across the top of the cylindrical body 101 when the device 100 is laid flat on a surface. In other words, when muscle is being rolled over the device 100 with a flat surface thereunderneath, only the light source elements 104 across the top of the body 101 are activated by the controller 103.

In embodiments, the device 100 may comprise a vibratory device 118 which may generate vibrations. The vibrations may be low-frequency and high-intensity to enhance massage of muscles. In this regard, the vibratory device 118 may comprise a relatively low-frequency electric motor turning an eccentric flyweight for deep muscle massage and/or a relatively higher frequency piezoelectric vibrator to encourage micro circulation of the skin.

In embodiments, the device 100 may comprise a heat source 119 which applies heat to muscle tissue via surface conduction. The heat source 119 may comprise an electroresistive heat source element drawing power from the battery 102. In embodiments, the body 101 comprises inlet and outlet vents forming a cooling duct therebetween. Furthermore, the body 101 may comprise a fan drawing air through the cooling duct to cool internal componentry, including the light source elements 104. In embodiments, the controller 103 may be operably coupled to a thermometer (not shown) to sense the internal temperature of the device to selectively activate the fan.

In embodiments, the light source elements 104 may be thermally coupled to a heat sink backing, such as one of aluminium and wherein the heat sink backing is thermally coupled to the cooling duct.

In embodiments, the device 100 comprises a data interface (not shown) for communicating with the mobile computing device. The mobile computing device may take the form of a mobile communication device executing a software application. The controller 103 may control the light source elements 104 according to data received via the data interface. The data interface may be a wireless data interface such as a Bluetooth data interface. Using the computing device, the user may be able to input user settings, select therapeutic programs and/or control the operation, duration and/or intensity of the light source elements 104.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. A massage device comprising a generally cylindrical body comprising an internal battery and controller for a plurality of light source elements, the body defining an exterior surface comprising the light source elements arranged in a matrix to emit light from a surface area of the surface.

2. The device as claimed in claim 1, wherein, the light source elements emit red and near infrared light in the range of 600 nm-1000 nm.

3. The device as claimed in claim 1, wherein the power density of the matrix of light source elements is between 0.005 W/cm² to 5 W/cm².

4. The device as claimed in claim 1, wherein the matrix of light source elements extends across more than 80% of the length of the body.

5. The device as claimed in claim 1, wherein the surface comprises a plurality of massage protrusions.

6. The device as claimed in claim 5, wherein the massage protrusions are flexible.

7. The device as claimed in claim 5, wherein at least a subset of the light source elements collocate with respective massage protrusions.

8. The device as claimed in claim 7, wherein at least one massage protrusion is generally shaped as a plano-convex lens.

9. The device as claimed in claim 5, wherein at least one massage protrusion is elongate with respect to a tangential axis of the body.

10. The device as claimed in claim 9, wherein at least one massage protrusion is domed with respect to a longitudinal axis of the body.

11. The device as claimed in claim 10, wherein the protrusions are arranged along a series of adjacent rings along the length of the body

12. The device as claimed in claim 11, wherein ends of protrusions of adjacent rings overlap.

13. The device as claimed in claim 5, wherein the device comprises a rigid cylindrical core and a transparent flexible sleeve.

14. The device as claimed in claim 13, wherein the sleeve comprises silicon.

15. The device as claimed in claim 13, wherein the light source elements are recessed in recesses in the cylindrical core.

16. The device as claimed in claim 15, wherein each recess is narrower than an adjacent massage protrusion

17. The device as claimed in claim 15, wherein the interior surface areas of the recesses comprise reflective material.

18. The device as claimed in claim 15, wherein the surface area of the cylindrical core away from the recesses comprises reflective material.

19. The device as claimed in claim 1, wherein the light source elements comprise subsets of light source elements emitting light in different wavelength rang es and wherein the controller can independently control the subsets to emit light in different wavelength ranges.

20. The device as claimed in claim 19, wherein the different wavelength ranges centre at approximately 660 nm and 850 nm respectively.

21. The device as claimed in claim 1, wherein the controller comprises a memory device comprising a therapeutic program encoded therein and wherein the controller controls the light source elements according to the therapeutic program.

22. The device as claimed in claim 21, wherein the therapeutic program specifies a dosage parameter.

23. The device as claimed in claim 22, wherein the controller controls at least one of duration and intensity according to the dosage parameter.

24. The device as claimed in claim 21, wherein the program further specifies at least one of wavelength range and treatment frequency parameters and wherein the controller is controllable to control the light source elements according to the parameters.

25. The device as claimed in claim 21, wherein the program comprises a user setting and wherein the controller is controllable to adjust the therapeutic program according to the user setting.

26. The device as claimed in claim 25, wherein the user setting comprises muscle group.

27. The device as claimed in claim 25, wherein the user setting comprises a user-specified level.

28. The device as claimed in claim 1, wherein the light source elements are independently controllable to emit light within radial angle ranges with respect to a longitudinal axis of the body.

29. The device as claimed in claim 28, wherein the controller interfaces a user control interface and wherein the control interface is configurable to specify an operative radial angle range.

30. The device as claimed in claim 28, wherein the device comprises at least one sensor operably coupled to the controller and wherein the controller determines an operative radial angle range in accordance with sensor data received from the at least one sensor.

31. The device as claimed in claim 28, wherein the at least one sensor is a pressure sensor operably coupled to the surface and wherein the controller is configured to control the light source elements to emit light at an operative radial angle of the surface where pressure is detected.

32. The device as claimed in claim 31, wherein the controller is further configured to control the light source elements across the length of the body depending on a location of pressure detected along the length of the body.

33. The device as claimed in claim 32, wherein the controller distinguishes between pressure applied by the flat surface and centrally applied pressure.

34. The device as claimed in claim 28, wherein the at least one sensor is an orientation sensor and wherein the controller is configured to control the light source elements to emit light dependent on the orientation of the cylindrical body.

35. The device as claimed in claim 34, wherein the controller is configured to control the light source elements to emit light across a top of the cylindrical body.

36. The device as claimed in claim 1, wherein the controller can control the intensity of the light source elements.

37. The device as claimed in claim 1, wherein the device further comprises a vibratory device.

38. The device as claimed in claim 37, wherein the vibratory device comprises an electric motor turning an eccentric flyweight.

39. The device as claimed in claim 37, wherein the vibratory device comprises a piezoelectric device.

40. The device as claimed in claim 1, wherein the device further comprises a heat source element.

41. The device as claimed in claim 40, wherein the heat source element is an electroresistive heat source element.

42. The device as claimed in claim 1, further comprising inlet and outlet vents forming and a cooling duct through the cylindrical body and a fan drawing air through the cooling duct.

43. The device as claimed in claim 42, wherein the light source elements are thermally coupled to a heatsink backing, and wherein the heatsink backing assembly coupled to the cooling duct.

44. The device as claimed in claim 1, further comprising a data interface for communicating with a mobile computing device and wherein the controller controls the light source elements according to data received via the data interface.

45. The device as claimed in claim 1, further comprising a user interface operably coupled to the controller.

46. The device as claimed in claim 45, wherein the user interface is located at an end of the cylindrical body.

47. The device as claimed in claim 46, wherein the user interface comprises a digital display.

48. The device as claimed in claim 1, wherein the cylindrical body comprises a diameter greater than 10 cm.

49. The device as claimed in claim 1, wherein the cylindrical body comprises a length greater than 20 cm.

50. The device as claimed in claim 1, wherein the cylindrical body comprises a length of approximately 30 cm.

51. The device as claimed in claim 1, wherein density of the light source elements increases towards the centre of the length of the body.

52. The device as claimed in claim 1, wherein the device comprises a pushbutton user interface which comprises an intensity control button to control light intensity.

53. The device as claimed in claim 1, wherein the device comprises a pushbutton user interface which comprises a timer control button to control and operational time of the light source elements.

54. The device as claimed in claim 1, wherein the device comprises a pushbutton user interface which comprises a vibration speed control button to control the speed of a vibratory device.

55. The device as claimed in claim 1, wherein the device comprises a pushbutton user interface which comprises a light frequency control button to control the intensity of light emitted by the light source elements.
